(19) United States
(12) Patent Application Publication
(10) Pub. No.: US 2026/0000082 A1
(43) Pub. Date: Jan. 1, 2026

(54) STABILIZED LIQUID HERBICIDE FORMULATION OF HIGH-LOAD PYRASULFOTOLE

(71) Applicant: ADAMA AGAN LTD., Ashdod (IL)

(72) Inventors: May LENIK, Gan Yavne (IL); Sergio NAHMOUD, Ashdod (IL)

(73) Assignee: ADAMA AGAN LTD., Ashdod (IL)

(21) Appl. No.: 18/869,092

(22) PCT Filed: May 29, 2023
(86) PCT No.: PCT/IL2023/050551

§ 371 Date: Nov. 25, 2024

Related U.S. Application Data
(60) Provisional application No. 63/346,920, filed on May 30, 2022.

(51) Int. Cl. A01N 43/56; A01N 25/02; A01N 25/32; A01P 13/02; C07C 317/04; C07D 231/20

(52) U.S. Cl. CPC A01N 43/56; A01N 25/02; A01N 25/32; A01P 13/02; C07C 317/04; C07D 231/20; C07B 2200/13

(57) ABSTRACT

The present invention relates to a stable and an efficient liquid herbicide formulation system comprising at least 125 g/L Pyrasulfotole based on total volume: an organic amine compound, and an organic solvent system; and to methods for controlling undesired plant species. The invention also relates to a new solid solvate of Pyrasulfotole.

### RELATED APPLICATION/S

This application claims benefits of U.S. Provisional Application No. 63/346,920 filed on May 30, 2022; the entire content of which is hereby incorporated by reference herein.

Throughout this application, various publications are cited. Disclosures of the documents and publications referred to herein are hereby incorporated in their entireties by references into this application.

### FIELD OF INVENTION

The present invention relates to a stable and an efficient liquid herbicide formulation system comprising at least 125 g/L Pyrasulfotole based on total volume; an organic amine compound, and an organic solvent system; and to methods for controlling undesired plant species. The invention also relates to a new solid solvate of Pyrasulfotole which was found to have formed with Dimethyl sulfoxide in absence of an organic amine in the formulation.

### BACKGROUND OF INVENTION

A large number of herbicidal active ingredients are known as inhibitors of the enzyme p-hydroxyphenylpyruvate dioxygenase (HPPD). As is the case with many other herbicidal active ingredients, these HPPD inhibitors are also not always sufficiently well tolerated by (i.e. not sufficiently selective in) some important crop plants such as corn, rice or cereals, so that their use is very limited. They can therefore not be employed in some crops, or only at such low application rates that the desired broad herbicidal activity against harmful plants is not ensured.

Pyrasulfotole is one such HPPD inhibitor effective against a wide range of broadleaf weeds found in fields of several types of crops. It is a bleaching herbicide, and inhibits the HPPD enzyme which impacts plant carotenoid pigment formation, and leading to chlorophyll degradation.

Like majority of herbicides, Pyrasulfotole in liquid formulations such as Emulsifiable concentrates (EC) are a common form of formulations as they can have a low production cost and involve simple technology. A liquid formulation may be tank mixed or otherwise combined with water and possibly other additives to form a field formulation. The field formulation can then be applied to crops by boom spraying and the like, in order to deliver the herbicide to the fields or crop.

Pyrasulfotole was first disclosed in Bayer's publication of WO01/74785. There were many types of liquid formulation of Pyrasulfotole commercially available which are suited for selective control of weeds in crops. The effectiveness of herbicides like Pyrasulfotole often depends on the concentration of the active Pyrasulfotole in the formulation system and also on its stability while storing and applying on the field.

Formulating a commercially acceptable Pyrasulfotole containing herbicide is fraught with many challenges. Pyrasulfotole is highly soluble in water but not so in organic solvents. When formulating compositions containing multiple chemicals including active compounds it is quite natural that physical and biological incompatibility would occur. To choose right blend of substances in a liquid formulation to incorporate high concentrate of Pyrasulfotole remains a big challenge in the art.

There have been lot of endeavors in the relevant art to prepare effective liquid formulations of Pyrasulfotole with high concentration. Various surfactant systems along with solvent systems have been attempted. However, no prior art has ever addressed the problem in the formulation to have a solvate deposition of Pyrasulfotole in a high dissoluting solvent, and solving the problem to get a very high concentrate of Pyrasulfotole liquid formulation.

In light of the above, there is endeavor in the present invention for a novel stable and an efficient liquid high concentrate herbicide formulation system of Pyrasulfotole, overcoming the solvate deposition, thereby enhancing overall efficiency of the formulation.

### SUMMARY OF THE PRESENT SUBJECT MATTER

We have reasonably addressed the challenges as described above as a whole or in part by arriving at a novel stable herbicide formulation system with suitable coformulants and an inventive solvent-surfactant system therein, as defined below.

The present invention relates to a liquid herbicide formulation system comprising (a) at least 125 g/L Pyrasulfotole based on total volume of formulation; (b) an effective amount of an organic amine compound; and (c) an effective amount of an organic solvent.

The present invention relates to a liquid herbicide formulation system comprising (a) at least 125 g/L Pyrasulfotole based on total volume of formulation; (b) an effective amount of an organic amine compound; (c) an effective amount of an organic solvent comprising Dimethyl sulfoxide, Cyclohexanone, Propylene carbonate, aromatic solvents, vegetable oils, and a solvent system thereof.

The present invention relates to a liquid herbicide formulation system comprising (a) at least 180 g/L Pyrasulfotole based on total volume of formulation; (b) an effective amount of an organic amine compound; (c) an effective amount of an organic solvent comprising Dimethyl sulfoxide, Cyclohexanone, Propylene carbonate, aromatic solvents, vegetable oils, and a solvent system thereof.

The present invention relates to a liquid herbicide formulation system comprising (a) at least 250 g/L Pyrasulfotole based on total volume of formulation; (b) an effective amount of an organic amine compound; (c) an effective amount of an organic solvent comprising Dimethyl sulfoxide, Cyclohexanone, Propylene carbonate, aromatic solvents, vegetable oils, and a solvent system thereof.

The present invention relates to a liquid herbicide formulation system comprising (a) at least 350 g/L Pyrasulfotole based on total volume of formulation; (b) an effective amount of an organic amine compound; (c) an effective amount of an organic solvent comprising Dimethyl sulfoxide, Cyclohexanone, Propylene carbonate, aromatic solvents, vegetable oils, and a solvent system thereof.

The present invention also relates to a liquid herbicide formulation system further comprising an effective amount of a safener selected from a group comprising Mefenpyr-diethyl, Cloquintocet-mexyl, and Isoxadifen-ethyl, and an effective amount of agriculturally acceptable carriers.

More specifically, present invention relates to a liquid herbicide formulation system comprising (a) at least 125 g/L Pyrasulfotole based on total volume of formulation; (b) an effective amount of an organic amine compound which is selected from a group comprising an organic amine alkoxylate and an alkyl alcohol amine; (c) an effective amount of an organic solvent which is Dimethyl sulfoxide.

More specifically, present invention relates to a liquid herbicide formulation system comprising (a) at least 125 g/L Pyrasulfotole based on total volume of formulation; (b) an effective amount of an organic amine compound which is an organic amine alkoxylate; (c) an effective amount of an organic solvent which is Propylene carbonate.

More specifically, present invention relates to a liquid herbicide formulation system comprising (a) at least 125 g/L Pyrasulfotole based on total volume of formulation; (b) an effective amount of an organic amine compound which is an alkyl alcohol amine; (c) an effective amount of an organic solvent which is Propylene carbonate.

More specifically, present invention relates to a liquid herbicide formulation system comprising (a) at least 125 g/L Pyrasulfotole based on total volume of formulation; (b) an effective amount of an organic amine compound which is an organic amine alkoxylate; (c) an effective amount of an organic solvent which is an aromatic solvent.

More specifically, present invention relates to a liquid herbicide formulation system comprising (a) at least 125 g/L Pyrasulfotole based on total volume of formulation; (b) an effective amount of an organic amine compound which is an alkyl alcohol amine; (c) an effective amount of an organic solvent which is an aromatic solvent.

The present invention also provides a method of treating a plant against undesired vegetation comprising applying herbicide formulation system disclosed herein, after dilution with water, to the target sites of the plant, so as to treat the plant against undesired vegetation.

The present invention also provides a process for the preparation of the herbicide formulation system disclosed herein from individual component parts.

The present invention further provides a method of controlling undesired vegetation comprising applying to the locus the herbicide formulation system disclosed herein. The present invention also provides a method for controlling grassy and broadleaf weeds comprising diluting with water herbicide formulation system disclosed herein, and applying the aqueous formulation to the locus of the plant.

The present invention provides a method of controlling undesired vegetation comprising applying to the locus liquid herbicide formulation system comprising (a) at least 125 g/L Pyrasulfotole based on total volume of formulation; (b) an effective amount of an organic amine compound; and (c) an effective amount of an organic solvent.

The present disclosure also includes a solid state form of Dimethyl sulfoxide solvate of Pyrasulfotole designated as Form B, and can be characterized by an XRPD pattern having peaks at 6.1, 11.2, 11.6, 13.4 and 22.5 degrees 2-theta±0.2 degrees 2-theta

### BRIEF DESCRIPTION OF THE FIGURES

FIG. 1 shows an X-ray powder diffractogram (XRPD) of Form B of Dimethyl sulfoxide (DMSO) solvate of Pyrasulfotole.

FIG. 2 shows an X-ray powder diffractogram (XRPD) of Form A of anhydrous Pyrasulfotole, following the process as disclosed in the US2002065200 publication.

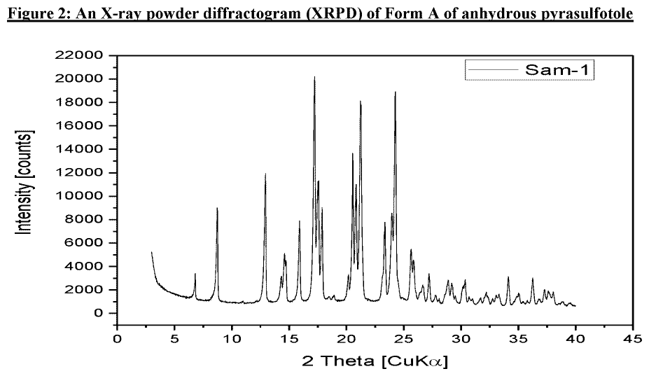

### DETAILED DESCRIPTION OF THE PRESENT SUBJECT MATTER

### Definitions

Prior to setting forth the present subject matter in detail, it may be helpful to provide definitions of certain terms to be used herein. Unless defined otherwise, all technical and scientific terms used herein have the same meaning as is commonly understood by one of skill in the art to which this subject matter pertains.

Certain exemplary embodiments are described to provide an overall understanding of the principles of the invention disclosed herein. It is assumed that those skilled in the art will understand that the inventive features and methods specifically described herein and illustrated in the experimental section are non-limiting exemplary embodiments and that the scope of the present invention is defined solely by the claims. The features illustrated or described in one exemplary embodiment may be combined with the features of other embodiments. Such modifications and variations are intended to be included within the scope of the present invention.

Reference will now be made in detail to embodiments, which are illustrated in the subsequent paragraphs, wherein reference numerals refer to like elements throughout. In this regard, the present embodiments may have different forms and should not be construed as being limited to the descriptions set forth herein. Accordingly, the embodiments are merely described below to explain aspects of the present disclosure.

As used herein, the term “pesticide” in general broadly refers to an agent that can be used to control and/or kill a pest. The term is understood to include but is not limited to fungicides, insecticides, nematicides, herbicides, acaricides, parasiticides or other control agents. For chemical classes and applications, as well as specific compounds of each class, see “The Pesticide Manual Thirteenth Edition” (British Crop Protection Council, Hampshire, UK, 2003), as well as “The e-Pesticide Manual, Version 3” (British Crop Protection Council, Hampshire, UK, 2003-04), the contents of each of which are incorporated herein by reference in their entirety.

As used herein, the term “system” means but is not limited to an assemblage of active ingredients and/or acceptable coformulants for application either by simultaneous, contemporaneous and/or succession application. It also means a combination in any time of application of the individual components e.g. succession and/or in any physical form, e.g. blend, solution, suspension, dispersion, emulsion, alloy, or the like. “System” also may refer to combining and applying the active components and acceptable coformulants as one composition and/or formulating each of the active component in the mixture or combination as separated compositions and application at the same time or in separated applications at the same time or different times.

The admixture or individual components may be in any physical form, e.g. blend, solution, suspension, dispersion, emulsion, alloy, or the like.

As used herein, the term “effective amount” refers to an amount of the active component that is commercially recommended for use to control weed. The commercially recommended amount for each active component, often specified as application rates of the commercial formulation, may be found on the label accompanying the commercial formulation. The commercially recommended application rates of the commercial formulation may vary depending on factors such as the plant species and the weeds to be controlled.

As used herein, the term “more effective” includes, but is not limited to, increasing efficacy of weed control, prolonging protection and reducing the amount of time needed to achieve a given level of weed control, prolonging the duration of protection against weeds after application and extending the protection period against weeds and/or reducing the amount of time needed to achieve a level of weed control compared to the herbicide formulation without suitable coformulants therein.

As used herein, the term “effective” when used in connection with any mixture or formulation system may be but is not limited to increase in controlling weeds, decrease time for effective controlling weeds, decrease the amount of the herbicide(s) which is required for effective controlling weed, extend the controlling effect of the individual herbicide active in the mixture in terms of type of crop and weed, prolong the time of controlling effect of the formulation. In particular, the term “effective” may refer to, increasing efficacy of weed control in untreated plant area, reducing the amount of time needed to achieve a given level of weed control, extending the protection period against weed and/or reducing the amount of time needed to achieve a level of weed control.

As used herein, the term “effective amount” refers to an amount of the individual components in an agrochemical formulation system or of the mixture which is critical for manufacturing effective formulation as well as for controlling harmful weed on crop plants and does not cause any significant damage to the treated crop plants.

As used herein, the term “agriculturally acceptable carrier” means carriers which are known and accepted in the art for the formation of compositions for agricultural or horticultural use.

As used herein, the term “coformulants” is defined as any substance that itself is not an active ingredient but is added to the composition or formulation such as additives, thickening agent, sticking agents, wetting agent, surfactants, anti-oxidation agent, anti-foaming agents and thickeners.

As used herein the term “plant” or “crop” includes reference to agricultural crops including field crops (soybean, maize, wheat, rice, etc.), vegetable crops (potatoes, cabbages, etc.), fruits (peach, etc.), semi-perennial crops (sugarcane) and perennial crops (coffee and guava).

As used herein the term “plant” or “crop” includes reference to whole plants, plant organs (e.g. leaves, stems, twigs, roots, trunks, limbs, shoots, fruits etc.), plant cells, seedling or plant seeds. This term also encompasses plant crops such as fruits.

The term “plant” may also include the propagation material thereof, which may include all the generative parts of the plant such as seeds and vegetative plant material such as cuttings and tubers, which can be used for the multiplication of the plant. It may also include spores, corms, bulbs, rhizomes, sprouts, basal shoots, stolons, and buds and other parts of plants, including seedlings and young plants, which are to be transplanted after germination, rooting or after emergence from soil or any other kind of substrate, be it artificial or natural.

As used herein, the term “locus” includes not only areas where weeds may already be shown, but also areas where undesired vegetation have yet to show and also area under cultivation. Locus include but is not limited to soil and other plant growth medium.

As used herein, the terms “control” or “controlling” or “treating” refers but is not limited to preventing weeds, protecting plants from weeds, delaying the onset of undesired vegetation, and combating weeds.

The term “applying” or “application”, as used herein, refers but is not limited to applying the compounds and compositions of the invention to the plant, to a site of weeds, to a potential site of weeds, which may require protection from undesired vegetation, or the environment around the habitat or potential habitat of the weeds. The application may be by methods described in the present invention such as by spraying, dipping, etc.

Throughout the application, descriptions of various embodiments the term “comprising” is used; however, it will be understood by one of skill in the art, that in some specific instances, an embodiment can alternatively be described using the language “consisting essentially of” or “consisting of.” The term “a” or “an” as used herein includes the singular and the plural, unless specifically stated otherwise. Therefore, the terms “a,” “an”, “one or more” or “at least one” can be used interchangeably in this application.

Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In this regard, use of the term “about” herein specifically includes ±10% from the indicated values in the range. In addition, the endpoints of all ranges directed to the same component or property herein are inclusive of the endpoints, are independently combinable, and include all intermediate points and ranges.

As used herein, the term “and/or” includes any and all combinations of one or more of the associated listed items. Expressions such as “at least one of,” when preceding a list of elements, modify the entire list of elements and do not modify the individual elements of the list.

A crystal form may be referred to herein as being characterized by graphical data as depicted in a Figure. Such data include, for example, powder X-ray diffractograms and solid state NMR spectra. As is well-known in the art, the graphical data potentially provides additional technical information to further define the respective solid state form which can not necessarily be described by reference to numerical values or peak positions alone. In any event, the skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Pyrasulfotole referred to herein as being characterized by graphical data “as depicted in” a Figure will thus be understood to include any crystal form of Pyrasulfotole, characterized with the graphical data having such small variations, as are well known to the skilled person, in comparison with the Figure.

A pure solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% or less, about 10% or less, about 5% or less, about 2% or less, about 1% or less, or 0% of any other forms of the subject compound as measured, for example, by XRPD. Thus, the solid state form of Pyrasulfotole described herein as substantially free of any other solid state forms would be understood to contain greater than about 80% (w/w), greater than about 90% (w/w), greater than about 95% (w/w), greater than about 98% (w/w), greater than about 99% (w/w), or 100% of the subject solid state form of Pyrasulfotole. Accordingly, in some embodiments of the disclosure, the described solid state form of Pyrasulfotole may contain from about 1% to about 20% (w/w), from about 5% to about 20% (w/w), or from about 5% to about 10% (w/w) of one or more other solid state forms of the same Pyrasulfotole.

In an aspect the present disclosure contemplates that a certain solid state form of Pyrasulfotole can exist in the presence of any other of the solid state forms or mixture thereof. Accordingly, in one embodiment, the present disclosure provides form B, for example, wherein form B is present in a solid form that includes less than 95%, less than 90%, less than 80%, less than 70%, less than 60%, less than 50%, less than 40%, less than 30%, less than 20%, less than 10%, less than 5%, less than 3% or less than 1% by weight of any other physical forms of Pyrasulfotole. The solid state forms of Pyrasulfotole as characterized herein may be present also in traces in an agrochemical compositions or formulations.

As used herein, unless stated otherwise, XRPD peaks reported herein are optionally measured using CuKα, radiation, λ=1.54187 Å.

As used herein, the term “isolated” in reference to solid state forms of Pyrasulfotole of the present disclosure corresponds to solid state form of Pyrasulfotole that is physically separated from the reaction mixture in which it is formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, about 10 to about 18 hours, or about 16 hours.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding methyl tert-butyl ether (MTBE) (1.5 v/v) to a 100 ml reaction mixture would indicate that 150 mL of MTBE was added.

As used herein, the term “reduced pressure” refers to a pressure of about 10 mbar to about 50 mbar.

For purposes of better understanding the present teachings and in no way limiting the scope of the teachings, each numerical parameter should be construed in light of the number of reported significant digits and by applying ordinary rounding techniques. In addition, the endpoints of all ranges directed to the same component or property herein are inclusive of the endpoints, are independently combinable, and include all intermediate points and ranges.

### Herbicide Formulations

There is always a challenge to make a liquid herbicide formulation system comprising high concentrate Pyrasulfotole, for increasing the desirable effect on the target surface of plant, resulting enhancement of overall efficiency of the formulation.

In this endeavor, the inventors have attempted several options to make a stable and efficient liquid formulation system by judiciously choosing conglomerate of coformulants which may result in creating a high concentrate Pyrasulfotole formulation without any deposit of solid side product. Several combinations of coformulants, solvents and organic amines were tried, and eventually the desired results were arrived at.

Several small experimentations were carried out to ascertain the solubilities of Pyrasulfotole in different solvents and it has been surprisingly found that an organic amine, for example, Genamin C-50 (a product of Clariant) plays a critical role in increasing the solubility Pyrasulfotole in respective solvents and adjuvant. Some of the results are tabulated as follows:

### Experiment 1:

Solubility of Pyrasulfotole in Dimethyl sulfoxide (DMSO) and in a mixture of Pyrasulfotole with DMSO and amine at room temp (24° C.) was tested.

| | | Solubility |
|---|---|---|
| Solvent | Rate | (%) |
| DMSO | 100 | 26.98 |
| DMSO/GENAMIN C-50 | 66/34 | 39.24 |

The solubility of Pyrosaflutole was increased by about 26%.

### Experiment 2:

Solubility of Pyrasulfotole in Cyclohexanone and in a mixture of Pyrasulfotole with Cyclohexanone and amine at room temp (24° C.) was tested.

| | | Solubility |
|---|---|---|
| Solvent | Rate | (%) |
| Cyclohexanone | 100 | 8.24 |
| Cyclohexanone/Genamin C-50 | 66/34 | 32.99 |

The solubility of Pyrosaflutole was increased by about 400%

### Experiment 3:

Solubility of Pyrasulfotole in Propylene carbonate and in a mixture of Pyrasulfotole with Propylene carbonate and amine at room temp (24° C.) was tested.

| | | Solubility |
|---|---|---|
| Solvent | Rate | (%) |
| Propylene carbonate | 100 | 4.59 |
| Propylene carbonate/Genamin C-50 | 70/30 | 28.57 |

The solubility of Pyrosaflutole was increased by about 620%

In an embodiment, the present invention relates to a liquid herbicide formulation system comprising (a) at least 125 g/L Pyrasulfotole based on total volume of formulation; (b) an effective amount of an organic amine compound; and (c) an effective amount of an organic solvent.

In an embodiment, the present invention relates to a liquid herbicide formulation system comprising (a) at least 180 g/L Pyrasulfotole based on total volume of formulation.

In an embodiment, the present invention relates to a liquid herbicide formulation system comprising (a) at least 250 g/L Pyrasulfotole based on total volume of formulation.

In specific embodiment, the present invention relates to a liquid herbicide formulation system comprising (a) at least 350 g/L Pyrasulfotole based on total volume of formulation.

In an embodiment, the present invention further relates to a liquid herbicide formulation system comprising an effective amount of a safener selected from a group comprising Mefenpyr-diethyl, Cloquintocet-mexyl, and Isoxadifen-ethyl; and an effective amount of agriculturally acceptable carriers.

In an embodiment, the present invention relates to a liquid herbicide formulation system comprising an effective amount of an organic amine compound which is selected from a group comprising an organic amine alkoxylate and an alkyl alcohol amine.

In a specific embodiment, the present invention relates to a liquid herbicide formulation system comprising an effective amount of an organic amine compound which is a fatty amine ethoxylate.

In a more specific embodiment, the present invention relates to a liquid herbicide formulation system comprising an effective amount of an organic amine compound which is Genamin C 50.

In a specific embodiment, the present invention relates to a liquid herbicide formulation system comprising an effective amount of an organic amine compound which is an alkyl alcohol amine.

In a more specific embodiment, the present invention relates to a liquid herbicide formulation system comprising an effective amount of an organic amine compound which is Ethanol amine.

In a more specific embodiment, the present invention relates to a liquid herbicide formulation system comprising an effective amount of an organic amine compound which is Tri-isopropanol amine.

In an embodiment, the present invention relates to a liquid herbicide formulation system comprising an effective amount of an organic solvent which is selected from a group comprising Dimethyl sulfoxide (DMSO), Cyclohexanone, Propylene carbonate, aromatic solvents, vegetable oils, and a solvent system thereof.

In a specific embodiment, the present invention relates to a liquid herbicide formulation system comprising an effective amount of an organic solvent which is Dimethyl sulfoxide.

In another specific embodiment, the present invention relates to a liquid herbicide formulation system comprising an effective amount of an organic solvent which is Propylene carbonate.

In another embodiment, the present invention relates to a liquid herbicide formulation system comprising agriculturally acceptable carriers which include, but not limited to an adjuvant and antifoam agent.

In an embodiment, the present invention provides a method of treating a plant against undesired weeds comprising applying herbicide formulation system as discussed herein, after dilution with water, to the target sites of the plant, so as to treat the plant against undesired weeds.

In an embodiment, the present invention provides a method of treating a plant against undesired weeds, wherein the undesired weeds are selected from a group comprising grassy and broadleaf weeds.

In an embodiment, the present invention provides a use of the herbicide formulation system as discussed herein, for controlling grassy and broadleaf weeds.

In an embodiment, the present invention provides a use of the herbicide formulation system as discussed herein, wherein grassy and broadleaf weeds are selected from a group comprising Echinochloa colonum, Alternenthera sessilis, Commelina communis, Eclipta alba., Ludvigia spp.

In an aspect of embodiment, this present invention also relates to a solid state form of Dimethyl sulfoxide (DMSO) solvate of Pyrasulfotole, which is formed during development of formulation involving DMSO.

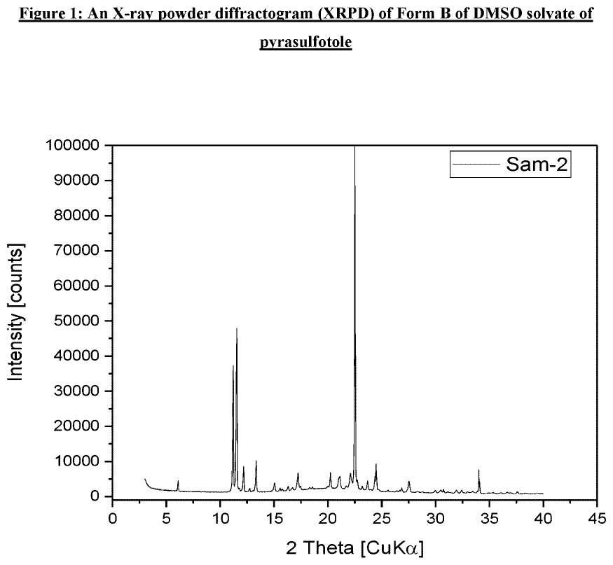

In another aspect of embodiment, this present invention also relates to a solid state form of Dimethyl sulfoxide (DMSO) solvate of Pyrasulfotole designated as Form B, and can be characterized by an XRPD pattern having peaks at 6.1, 11.2, 11.6, 13.4 and 22.5 degrees 2-theta±0.2 degrees 2-theta, an XRPD pattern as depicted in FIG. 1; and combinations of these data.

In an embodiment, crystalline Form B of DMSO solvate of Pyrasulfotole may be characterized by any combination of at least four peaks in an XRPD pattern as depicted in FIG. 1.

The following examples illustrate the practice of the present invention in some of its embodiments but should not be construed as limiting the scope of the invention. Other embodiments will be apparent to one skilled in the art from consideration of the specification and examples. It is intended that the specification, including the examples, is considered exemplary only without limiting the scope and spirit of the invention.

### Preparatory Examples

Various methods were tried to make high concentrate Pyrasulfotole in a liquid formulation. Inventors have eventually zeroed in to Dimethyl sulfoxide (DMSO) as solvent. Following results were observed on dissolution pattern Pyrasulfotole in DMSO:

| | Results |
|---|---|
| 38 g DMSO + 17 g Pyrasulfotole | Turbid |
| | (insoluble crystals) |
| 25 gr DMSO + 13 gr Genamin | Clear solution |
| C-50 + 17 g Pyrasulfotole | |
| 25 gr DMSO + 13 gr | Clear solution |
| EthanolAmine + 17 g Pyrasulfotole | |
| 25 gr DMSO + 13 gr Genamin | Clear solution |
| O-50 + 17 g Pyrasulfotole | |

It is clearly seen that the addition of an organic amine makes the clear solution and in absence of that amine surprisingly make the solution turbid with the formation insoluble crystals. The problem of this turbidity due to these insoluble crystals was solved by adding effective amount of an organic amine (e.g. Genamin C-50, Ethanol Amine, Tri-isopropanol amine, Genamin O-50).
The insoluble crystals were identified as Pyrasulfotole-Dimethyl sulfoxide solvate. In order to identify the physical characteristics of this Pyrasulfotole-Dimethyl sulfoxide solvate the sample was separately prepared and XRPD data was generated.

### Analytical Methods

### X-Ray Powder Diffraction Method:

Powder X-ray Diffraction was performed on an X-Ray powder diffractometer PanAlytical X'pert Pro; CuKα, radiation, (λ=1.54187 Å); zero background sample holders.

### Measurement Parameters:

Scan range: 4-40 degrees 2-theta
Scan speed 3°/min (for Form C, Form D); and 1.2°/min (for Form F)
Sample holder: Zero background silicon plate
   Prior to analysis, the samples were gently ground using a mortar and pestle to obtain a fine powder. Optionally, silicon powder can be added in a suitable amount as internal standard in order to calibrate the positions of the diffractions. The ground sample was adjusted into a cavity of the sample holder and the surface of the sample was smoothed using a cover.

### Example

Solid form of Pyrasulfotole-Dimethyl sulfoxide solvate was prepared according to the following procedures:
Pyrasulfotole tech was prepared following US2002065200 publication.
32 g of Pyrasulfotole tech was added to 68 g of Dimethyl sulfoxide (DMSO) in a flask. The flask is stirred until dissolution of the compound is completed. The stirring is continued until a precipitated solid is observed in the solution. The solid was filtered and dried in a vacuum. Solid was then gently ground with spatula and analysed by XRD. The XRPD data is generated which is demonstrated in the FIG. 1.

### Formulations:

Exemplary embodiments of the formulation of the present invention are illustrated in the below Tables:

TABLE 1
Pyrasulfotole 350 SL

g/1 L

a.

| | |
|---|---|
| Pyrasulfotole | 350 |
| Dimethyl sulfoxide (DMSO) | 810 |

b.

| | |
|---|---|
| Pyrasulfotole | 350 |
| Dimethyl sulfoxide (DMSO) | 636 |
| Genamin C-50 | 174 |

This formulation is unstable because of precipitation of the Pyrasulfotole solvate
This above formulation is found to contain no trace of solid Pyrasulfotole DMSO solvate crystals.

TABLE 2
Pyrasulfotole 180 SL

g/1 L

c. Unstable formulation at room temperature

| | |
|---|---|
| Pyrasulfotole | 180 |
| Propylene | 1020 |
| carbonate | |

d. Stable composition at room temperature

| | |
|---|---|
| Pyrasulfotole | 180 |
| Propylene | 840 |
| carbonate | |
| Genamin C-50 | 180 |

A specific composition embodying the present invention is disclosed in the following table:

TABLE 3
Emulsifiable concentrate (EC) Formulation:

| Ingredient | CAS No. | Chemical Name | Function | v/w | w/w |
|---|---|---|---|---|---|
| Pyrasulfotole | 365400-11-9 | 2-(Methylsulfony1)-4- | Herbicide | 230 | 21.1 |
| techical | | (trifluoromethyl)benzoic acid | | | |
| BYK-024 | 25322-69-4 | (A) Polypropylene glycol | Defoamer | 1.6 | 0.15 |
| | | B) Polysiloxanes | | | |
| Dimethyl Sulfoxide | 67-68-5 | Dimethyl sulfoxide | Solvent | 288 | 26.42 |
| (DMSO) | | | | | |
| Emulsogen EL 360 | 61791-12-6 | Ethoxylated vegetable oil | Emulsifier | 45 | 4.13 |
| Genamin C 050 | 61791-14-8 | Coconut fatty amine | | 250 | 22.94 |
| | | ethoxylate 5-8 EO | | | |
| Mefenpyr Diethyl | 135590-91-9 | diethyl (RS)-1-(2,4- | Safener | 58 | 5.32 |
| | | dichlorophenyl)-5-methyl-2- | | | |
| | | pyrazoline-3,5-dicarboxylate | | | |
| Nansa_EVM70/2E | 90194-26-6, | (A)Benzenesulfonic acid, | Surfactant | 15 | 1.38 |
| | 26264-06-2, | C10-13-alkyl derivs, calcium | | | |
| | 68584-23-6, | salt, (B)2-ethylhexane-1-ol | | | |
| | 84989-14-0, | | | | |
| | 104-76-7 | | | | |
| Propylene glycol | 107-98-2, | 1-methoxypropan-2-ol, | Surfactant | 47 | 4.31 |
| monomethyl ether | 1589-47-5 | 2-ethoxypropanol | | | |
| Silicaid AF-100 | 63148-62-9 | Dimethyl siloxane | Antifoam | 0.1 | 0.0092 |
| Agnique ME 18 | 67762-38-3 | C16-18-unsaturated fatty | Adjuvant | Up to | Up to |
| RD-F | | acid methyl esters | | 1000 L | 100% |

The process of preparation of this formulation is summarized as follows:
Agnique ME 18 RD-F, Propylene glycol monomethyl ether and Dimethyl Sulfoxide (DMSO) are charged in a vessel. Thereafter, BYK-024, Emulsogen EL 360, Genamin C 050 and Nansa_EVM70/2E are added under agitation. The vessel is put under continuous stirring followed by addition of Pyrasulfotole tech and Mefenpyr Diethyl. All the materials are mixed thoroughly until homogeneity.
While the present disclosure of the invention may be susceptible to various modifications and alternative variations, specific embodiments have been described by way of examples in detail herein. However, it is understood that the present disclosure is not intended to be limited to only particular forms as disclosed. Rather, the present disclosure is to cover all modifications, equivalents, and alternatives falling within the scope of the present disclosure as defined by the following claims and their legal equivalents.

What is claimed is:

1. A liquid herbicide formulation system comprising:
(a) at least 125 g/L Pyrasulfotole based on total volume of formulation;
(b) an effective amount of an organic amine compound;
(c) an effective amount of an organic solvent.

2. The liquid herbicide formulation system of claim 1, wherein the organic amine compound is selected from a group comprising an organic amine alkoxylate and an alkyl alcohol amine.

3. The liquid herbicide formulation system of any of claim 1 or 2, wherein the organic amine alkoxylate compound is a fatty amine ethoxylate, which is Genamin C 50.

4. The liquid herbicide formulation system of any of claims 1 to 2, wherein the alkyl alcohol amine compound is selected from a group comprising Ethanol amine and Tri-isopropanol amine.

5. The liquid herbicide formulation system of any of claims 1 to 4, wherein the organic solvent is selected from a group comprising Dimethyl sulfoxide (DMSO), Cyclohexanone, Propylene carbonate, aromatic solvents, vegetable oils, and a solvent system thereof.

6. The liquid herbicide formulation system of any of claims 1 to 5, wherein the organic solvent is DMSO or a solvent system thereof.

7. The liquid herbicide formulation system of claim 5, wherein the organic solvent is an aromatic solvent.

8. The liquid herbicide formulation system of claim 5, wherein the organic solvent is Propylene carbonate.

9. The liquid herbicide formulation system of claim 5, wherein the vegetable oils are selected from a group comprising rapeseed oil.

10. The liquid herbicide formulation system of any of claims 1 to 6, wherein the formulation system is free of a solid form of Pyrasulfotole DMSO solvate.

11. The liquid herbicide formulation system of any of claims 1 to 10, wherein herbicide formulation system comprising (a) at least 180 g/L Pyrasulfotole based on total volume of formulation.

12. The liquid herbicide formulation system of any of claims 1 to 10, wherein herbicide formulation system comprising (a) at least 250 g/L Pyrasulfotole based on total volume of formulation.

13. The liquid herbicide formulation system of any of claims 1 to 10, wherein herbicide formulation system comprising (a) at least 350 g/L Pyrasulfotole based on total volume of formulation.

14. The liquid herbicide formulation system of any of claims 1 to 13, wherein herbicide formulation system further comprising an effective amount of a safener and an effective amount of agriculturally acceptable carriers.

15. The liquid herbicide formulation system of claim 14, wherein the safener selected from a group comprising Mefenpyr-diethyl, Cloquintocet-mexyl, and Isoxadifen-ethyl.

16. A method of treating a plant against undesired weeds comprising applying herbicide formulation system of any one of claim 1-15, after dilution with water, to the target sites of the plant, so as to treat the plant against undesired weeds.

17. The method of claim 16, wherein the undesired weeds are selected from a group comprising grassy and broadleaf weeds.

18. Use of the herbicide formulation system of any one of claim 1-15, for controlling grassy and broadleaf weeds.

19. The use of the herbicide formulation system of claim 18, wherein grassy and broadleaf weeds are selected from a group comprising Echinochloa colonum, Alternenthera sessilis, Commelina communis, Eclipta alba., Ludvigia spp.

20. A solid state form of Dimethyl sulfoxide (DMSO) solvate of Pyrasulfotole

21. A Crystalline Form B of DMSO solvate of Pyrasulfotole of claim 20, which is characterized by data selected from one or more of the following:
i. an XRPD pattern having peaks at 6.1, 11.2, 11.6, 13.4 and 22.5 degrees 2-theta±0.2 degrees 2-theta;
ii. an XRPD pattern as depicted in FIG. 1.
